(19) United States
(12) Patent Application Publication
(10) Pub. No.: US 2004/0241256 A1
(43) Pub. Date: Dec. 2, 2004

(54) Medicinal compositions & therapeutic methods

(76) Inventors: Seymour Ehrenpreis, Skokle, IL (US); Lawrence Howard, Misdsion Viejo, CA (US)

Correspondence Address: John J Connors, Connors & Associates, 1600 Dove Street, #220, Newport Beach, CA, 92660, US

(21) Appl. No.: 10/497,631

(22) PCT Filed: Dec. 5, 2002
(86) PCT No.: PCT/US02/38898

(51) Int. Cl. A61K 35/78; A61K 31/7024; A61K 31/737; A61K 31/7076; A61K 38/05

(52) U.S. Cl. 424/734; 424/771; 514/18; 514/23; 514/46; 424/94.1; 514/62; 514/54; 514/2; 514/419; 514/567; 424/769; 424/94.65

(57) ABSTRACT

The compositions of this invention comprise a mixture of (1) phenylalanine and a dietary food supplement, (2) leucine and a dietary food supplement, and (3) hydrocinnamic acid and a dietary food supplement. The compositions are used for medicinal purposes to alleviate a variety of maladies.

### RELATED PATENT APPLICATIONS & INCORPORATION BY REFERENCE

This application is a PCT application based on U.S. provisional patent application Ser. No. 60/338,320 entitled “Anti-Inflammation/Analgesic Compositions & Methods Of treating Arthritis And Other Painful Conditions,” filed Dec. 6, 2001. This related application is incorporated herein by reference and made a part of this application. If any conflict arises between the disclosure of the invention in this PCT application and that in the related provisional application, the disclosure in this PCT application shall govern. Moreover, Applicants incorporate herein by reference any and all U.S. patents, U.S. patent applications, and other documents cited or referred to in this application or cited or referred to in the U.S. patents and U.S. patent applications incorporated herein by reference.

### DEFINITIONS

The words “comprising,” “having,” and “including,” and other forms thereof, are intended to be equivalent in meaning and be open ended in that an item or items following any one of these words is not meant to be an exhaustive listing of such item or items, or meant to be limited to only the listed item or items.

The term “arthritis” means inflammation of the articular extremity of a bone resulting in erosion of the cartilage, loss of range of motion and pain.

### BACKGROUND OF INVENTION

Phenylalanine is now shown by the inventors to be a medication for various maladies, including high blood pressure and pain relief, including treating arthritis. This amino acid includes D-phenylalanine (DPA) or D,L-phenylalanine (DLPA); the latter is a 50-50 mixture of the D and L forms of phenylalanine (DPA and LPA). Phenylalanine is considered to be a dietary supplement; DLPA is sold over-the-counter. DPA, but not LPA, is an inhibitor of enzymes that inactivate the naturally occurring peptides—the enkephalins and other endorphins; this compound is termed an enkephalinase inhibitor. Thus it is the DPA not LPA—the L form of phenylalanine—which is the active agent in providing relief in arthritis and other painful conditions. As a result of this activity, levels of enkephalins in particular have been shown to increase in the central nervous system in mice. As a result, various painful conditions are relieved. DPA has been shown to have another activity, namely, as an anti-inflammatory agent. These activities—analgesia plus anti-inflammatory activity—suggest efficacy of DPA or DLPA in arthritis; data indicating that this is indeed the case as presented below.

DPA also lowers above normal blood pressure. This is in contradistinction to the conventional anti-hypertensive drugs that can significantly lower blood pressure even if it is normal. The blood pressure-lowering effect of DPA is long-lasting in both animals and humans. Thus, DPA may be administered as a once-a-day agent to achieve a blood-pressure lowering effect. At the present time DPA appears to be unique among inhibitors of endorphin degradation since it was demonstrated that two other compounds with similar activities—thiorphan and actinonin—were essentially devoid of anti-hypertensive activities.

In general, the causes of arthritis are not known, particularly osteoarthritis. Rheumatoid arthritis appears to be an autoimmune disease. Arthritis in animals and humans is attributed to the presence of excessive amounts of prostaglandins and other products of the arachidonic acid cascade, and proteolytic enzymes elaborated during the disease process, is treated by means of combinations of drugs and natural products which serve to prevent the accumulation of pathophysiological factors. Also, various painful conditions in animals and humans are treated by raising the levels of endorphins in the central nervous system through these same means. A primary agent in all such combinations of drugs and natural products is phenylalanine, in doses sufficient to reverse, or prevent, the inflammation and pain of arthritis, or many other painful conditions.

The sequence of events that occur in an arthritic joint is as follows:

If a pathophysiological event occurs, e.g. trauma, joint infection, autoimmune response, the arachidonic acid cascade is activated resulting in the generation of prostaglandins and leukotrienes; these substances promote inflammation and pain. Cell membranes within the joint are damaged resulting in the release of proteolytic enzymes, which can erode the cartilage. Also present within joint synovial fluid are various endorphins (enkephalins, _endorphin); these are analgesic peptides. Under ordinary conditions, they would counteract the pain caused by the products of the arachidonic acid cascade. However, if the conditions persist, or are particularly severe, the condition becomes irreversible and full-blown arthritis ensues. The reasons for this are as follows:

The amount of proteolytic enzymes released becomes sufficient to degrade the joint cartilage causing its destruction. In addition, these enzymes degrade the endorphins present. Finally, there is a build-up of the products of the arachidonic acid cascade. The net result is continuing pain and inflammation, and loss of range of motion, i.e., the arthritic condition.

There are several classes of drugs, which, to a certain extent, can alleviate the painful condition as well as the inflammatory response. These include the following: Various steroids, aspirin and other non-steroidal anti-inflammatory drugs (NSAIDs) such as ibuprofen, indomethacin, naprosyn, sulindac, etc. These drugs block the arachidonic acid cascade thereby providing relief from the inflammation and pain caused by the prostaglandins and related compounds.

In addition, there are drugs which may slow down the progress of the disease; these are called DMARDs (disease modifying anti-arthritic drugs) and include methotrexate, cyclosporine, and various gold compounds. Unfortunately, all of these drugs have a serious drawback in that they may cause serious, even fatal, adverse reactions. In addition, these drugs are involved with many interactions with other drugs, such interactions at times being very deleterious, and requiring difficult adjustment of dosage for the interacting drugs.

More recently, several nutraceuticals have been introduced with some success for arthritis treatment. Among these are glucosamine (GS), chondroitin sulfate (CSA) and cetyl myristoleate. These naturally occurring compounds offer a new approach to treatment, namely, to slow down, or perhaps even reverse, the destruction of joint cartilage that is characteristic of arthritis. It should be noted that none of these newer agents have analgesic or anti-inflammatory activity.

### SUMMARY OF INVENTION

This invention, with its several desirable features, is summarized in the CLAIMS that follow. After reading the following section entitled “DETAILED DESCRIPTION OF SOME EMBODIMENTS OF THIS INVENTION,” one will understand how the features of this invention provide its benefits. The benefits of this invention include, but are not limited to, treating the following maladies: high blood pressure, osteoarthritis and rheumatoid arthritis, anxiety, depression, psychological disorders such as, for example, ADHD and ADD, OCD, stress, agorophobia, bulimia, anorexia, insomnia, lack of focus, craving including food, drug and alcohol addictions, fibromyalgia, and pain including (1) preventing or reversing the inflammation and chronic and acute pain of arthritis (2) reversing the destruction of cartilage that is present in arthritis, and (3) headache, toothaches, low back pain, musculoskeletal pain, pre-menstrual pain, pain due to sports injury, carpal tunnel injury, broken bones, post operative surgery, and dental pain.

The compositions of this invention comprise:

(1) a mixture of phenylalanine and the dietary food supplement identified below;

(2) a mixture of leucine and the dietary food supplement identified below;

(3) a mixture of hydrocinnamic acid and the dietary food supplement identified below; and

(4) a mixture of and the dietary food supplement identified below and a blend of any two of the phenylalanine, leucine, and hydrocinnamic acid of these ingredients.

The phenylalanine, leucine, hydrocinnamic, or mixtures thereof, may be present in an amount of from 5 to 95 weight percent and the dietary food supplement may be present in an amount of from 5 to 95 weight percent. The phenylalanine may include as the major component the D-phenylalanine, and the leucine may include as the major component the D-leucine.

The dietary food supplements consist of glucosamine, chondroitin sulfate, Cat's claw, Devil's claw, cetyl myristoleate, a mixture of a cetyl ester and cetyl myristoleate, coenzyme Q-10, fructose 1-6 diphosphate, glutathione (reduced), melatonin, Kava Kava extract, s-adenosylmethionine (SAMe), SAMe including as a major component the (SS)-(+)-SAMe, bromelain, a mixture of white willow bark powder and extract of salicin, either or both hydrolyzed and un-hydrolyzed Type II Collagen, methyl-sulfonyl-methane, hyaluronic acid, pine bark extract, Citrulline, L-tryptophine, Gingko Biloba, ginsing, St. John's Wort, creatine, Ribose, Ephedra, Ephedrine, glutamine, L-carnitine, Androstene compounds, Citicoline, NADH, B-Vitamins, Folic Acid, Biotin, Tyrosine, Vitamin C, Trimethylglycine, caffeine, protein powders, and mixtures thereof.

The various individual combinations of the above ingredients are each unique and some combinations are more suited to treating specific maladies than others as discussed subsequently. Many of the more effective compositions include S-adenosyl-L-methionine, and its salts, (either or both herein referred to as SAMe). These SAMe compounds are well known pharmacologically active compositions that combat, for example, depression, arthritis, and liver diseases such as, for example, cirrhosis. SAMe occurs as two diastereoisomers: (RS)-(+)-SAMe and (SS)-(+)-SAMe. The (SS)-(+)-SAM-e diastereoisomer is the pharmacologically active diastereoisomer. In one embodiment of this invention the SAMe includes as a major component the diastereoisomer (SS)-(+)-SAMe. For example, SAMe products containing (SS)-(+)-SAMe at a concentration of at least 95 weight percent (%) of the total diastereoisomers mixture are used. A suitable source of SAMe, including the SAMe with the higher concentration of the (SS)-(+)-SAMe diastereoisomer, Gnosis S. r.1. of Milan, Italy.

The compositions of this invention may include a therapeutic amount of an anti-depressant medicinal substance such as, for example, fluoxitine, anti-depressants in the SSRI class, or tricyclic anti-depressants. These anti-depressant medicinal substances are typically prescription drugs that have adverse side effects. Reduce dosages of these drugs are employed in the compositions of this invention.

The compositions of this invention are easily administered by the oral route. For example, oral administration via tablets (plain or enteric coated and/or time released), caplets (plain or enteric coated and/or time released), liquids, drinks (ready made or drinks by adding liquid), oral sprays and gels and soft gels (plain or enteric coated and/or time released). In addition to oral administration, the compositions of this invention may be administered by IV's, suppositories, creams, nasal sprays, inhalants, muscle/skeletal injections, added to foods, energy bars, etc., and patches. These compositions may be given in dosages that are safe and provide therapeutic efficacy at such dosages. For example, even trace amounts may be beneficial such as, for example, as low as 1 microgram. Typically, the dosage of the composition is from 1 to 400 grams per day, divided, preferably equally, into from 2 to 4 applications per day. In many instances, a dosage of 2-4 grams once a day is adequate.

### DETAILED DESCRIPTION OF SOME EMBODIMENTS OF THIS INVENTION

In one embodiment particularly suited for treating high blood pressure, the phenylalanine or leucine or hydrocinnamic acid (or mixtures of two or more) is mixed with Co-Q-10, or Citrulline, or L-Glutamine, or Glutathione, or L-Tryptophane, or mixtures thereof. In one embodiment particularly suited for treating high blood pressure, phenylalanine or leucine or hydrocinnamic acid is mixed with a therapeutic amount of an anti-hypertensive drug especially appropriate for this use. These compositions are especially useful when they include a dietary food supplement such as Co-Q-10. Examples of such anti-hypertensive drugs include blood vessel dilators such as diazoxide and diuretics such as a thiazide diuretic such as hydrochlorothiazide, a loop diuretic such as furosemide, a potassium-sparing diuretic such as triamterene, an adrenergic-beta-blocking agent a beta blocker such as propranolol, an angiotensin converting enzyme inhibitor such as lisinopril, an angiotensin II receptor, antagonist such as losartan, an alpha-blocker such as prazosin, a calcium channel blocker such as verapamil, a T-type calcium antagonist including for example₂ adrenergic agonist such as clonidine, an inhibitor of norepinephrine sythesis selected from the group consisting of methyl-p-tyrosine, diethyldithiocarbonate, inhibitors of dopamine-_-hydroxylase, a source of magnesium, Bestatin, Thiorphan.

In one embodiment particularly suited for reducing pain accompanying athletic exercise, improving endurance, strength, and performance, the phenylalanine or leucine or hydrocinnamic acid is mixed with Caffeine, Creatine, Ribose, Phosphates, Branch Chain Amino's, FDP, etc., strength and body building protein powders, Ephedra or Ephedrine, e. g., those found in herbs, Creatine, Glutamine, L-Carnitine, Androstene compounds or mixtures thereof.

In one embodiment particularly suited for improved brain functions, the phenylalanine or leucine or hydrocinnamic acid is mixed with Gingko Biloba, Ginsing, St. John's Wort, Huperzine, Phosphatidyl Choline, Phosphatidyl Serine, Wild Jujube Seed. Additionally, N-Acetyl-Glucosamine, Green Tea, Ginseng, Vitamin B-1,B-2, B-3, B-5, B-6, B-12, Folic Acid, Biotin, L-Tyrosine, Acetyl-L-Cysteine, Magnesium, Vitamin C may be added to this mixture.

In one embodiment particularly suited for improved mental health, with SAMe, Glutathione, Co-Q-10, Citicoline, NADH, B-Vitamins. Folic acid, Bioti, Tyrosine, GABA, L-Glutamine, Vitamin C, and Trimethylglycine.

In one embodiment for treating arthritis, a therapeutic amount of the primary agent phenylalanine is blended with a therapeutic amount of one or more of the dietary food supplements set forth in Table 7. In a second embodiment of this invention, one or more of the following: aspirin, acetaminophen, or a non-selective, non-steroidal, anti-inflammatory drug (NSAID), as well as COX 2 inhibitors (NSAID), is added to the blend of phenylalanine and dietary food supplement of the first embodiment. One especially beneficiary composition is a blend of phenylalanine, acetaminophen, and therapeutic amount of glutathione. One or more of the NSAIDs listed in Table 6 may be used. Preferably D-phenylalanine is used in both embodiments. In all these embodiments, the amount of D-phenylalanine administered is between approximately 10 and approximately 3000 mg/day, and the amount of D,L-phenylalanine administered is between approximately 20 and approximately 6000 mg/day. Administration may be in the form of a tablet or a capsule.

Each of the components of the proposed combinations provides some advantages over individual usage. DPA/DLPA provides relief of pain, as do the NSAIDs. The combination of NSAIDs with D-phenylalanine or D,L-phenylalanine has been shown to greatly enhance the analgesia provided by the NSAIDs alone. Thus, the combination provides pain relief for those who fail to respond, or respond inadequately, to D-phenylalanine or D,L-phenylalanine alone or NSAIDs alone. DPA/DLPA combined with the NSAIDs have potent anti-inflammatory activities. DPA/DLPA is extremely safe as shown both in animal and human studies. Glucosamine and chondroitin sulfate can promote regeneration of cartilage, which may be destroyed in arthritis. Thus, the combinations discussed above provide the following advantages over using them individually:

1. Greater efficacy for relief of pain.

2. Reduced drug toxicity, in particular by reducing the dosage of acetaminophen, aspirin and other NSAIDs.

3. Reduced incidence of drug-drug interactions due to the reduction in dosage of aspirin or other NSAIDs.

4. Reversal of inflammation.

5. Reversal of cartilage destruction.

6. Rebuilding of cartilage destroyed by the arthritic processes.

7. Prevention of recurrence of the disease.

One aspect of the present invention is to use phenylalanine, leucine or hydrocinnamic acid, or combinations of these ingredients, to inhibit the enzymes that inactivate certain endorphins, thereby permitting these endorphins to accumulate in the central nervous system and the synovial fluid of joints. In so doing, these endorphins relieve the unwanted symptoms. For example, when treating arthritis, the pain and inflammation are reduced by providing analgesia for painful conditions. The compositions of this invention may include NSAIDs known to be effective in treating pain and inflammation, namely, aspirin, ibuprophen, etc., permitting the use of lower doses of each component to achieve the desired effect. As a result, adverse effects as well as drug-drug interactions are reduced.

Another aspect of the present invention is to use phenylalanine, leucine or hydrocinnamic acid, or combinations of these ingredients, at a dosage sufficient to relieve pain and inflammation of arthritis and the pain that accompanies such conditions as headache, low back pain, musculoskeletal pain, dental pain and pre-menstrual pain.

All of the compounds and combinations described above can be offered as pharmaceutically accepted formulations using methods known to those of ordinary skill in the art. Some of these formulations may only be administered by the oral route, whether solely or in combination. The dosage of phenylalanine, leucine or hydrocinnamic acid, or combinations of these ingredients, or in combination with any of the other compounds, will depend on the severity of the arthritic, or other painful condition, as well as any existing or potential co-morbidity. It should be noted that the present invention has application for both human and veterinary use. Suggested oral dosages for humans are shown in Table 5. Formulations for human use will be in the form of tablets or capsules, normal and sustained release.

### BRIEF DESCRIPTION OF THE TABLES

Table 1: Anti-inflammatory activity of DPA using the rat paw carrageenan method.

Table 2: Anti-inflammatory activity of DPA using the mouse writhing test.

Table 3: Analgesic activity of DPA using the mouse hot plate method. Combination of DPA with indomethacin and sulindac, two NSAIDs.

Table 4: Efficacy of DPA in arthritis and a variety of other musculoskelatal diseases: human studies.

Table 5: Suggested dosage schedule for treating arthritis in humans using DPA or DLPA alone or in combination with various other compounds.

Table 6: List of NSAIDs, including COX 2 Inhibitors

Table 7: List of dietary food supplements combined with DPA

### DETAILED DESCRIPTION OF THE METHODS CITED ABOVE AND RESULTS OBTAINED

1. Anti-inflammatory activity of DPA using the rat paw carrageenan method.

This is a generally recognized method for evaluating anti-inflammatory drugs of the aspirin/NSAID type or those that counteract the action of the prostalandins. Such drugs are useful for treating conditions such as arthritis in animals and humans. In this method, the rat paw of the rat is injected with carrageenan, which causes swelling of the paw due to accumulation of fluid. A drug that prevents this swelling would be expected to have anti-inflammatory and hence anti-arthritic activity in humans. In this study, DPA was administered by various routes and times into groups of rats (6 male albino rats per group—weight approximately 250 grams) before carrageenan and the degree of swelling was measured by the extent of displacement of water when the paw was immersed into a calibrated cylinder. A placebo consisting of saline solution was similarly administered. Results are shown in Table 1.

TABLE 1
Anti-nflammatory activity of DPA using the
rat paw carrageenan method

% inhibition
of swelling

A. Subcutaneous injection

| | | |
|---|---|---|
| 120 minutes | Placebo | 0 |
| before | DPA 8 mg/kg | 15 |
| carrageenan | 125 mg/kg | 37 |
| | 500 mg/kg | 35 |
| 6 hours before | Placebo | 0 |
| carrageenan | DPA 62.5 | 35 |
| | 250 | 36 |
| | 500 | 75 |

B. Oral administration

| | | |
|---|---|---|
| 150 minutes | Placebo | 0 |
| before | DPA 125 mg/kg | 7 |
| carrageenan | 250 mg/kg | 10 |
| | 500 mg/kg | 24 |
| | 1000 mg/kg | 62 |
| | 1500 mg/kg | 79 |

2. Anti-inflammatory activity of DPA using the mouse phenylbenzylquinone (PBQ) writhing test.

This is a generally recognized method for evaluating anti-inflammatory drugs of the aspirin/NSAID type; those drugs which counteract the action of the prostaglandins. Such drugs are useful for treating conditions such as arthritis in animals and humans.

In this method a solution of PBQ is injected intra-peritoneally into groups of 6 mice (male, albino, 22-25 grams). Within a few minutes, the mice began to writhe. DPA was injected subcutaneously or given by mouth at different times prior to PBQ. The number of writhes were counted over a period of 20 minutes. The effects of DPA were compared to that of similarly administered saline control. Results are shown in Table 2. A drug that blocks writhing would be expected to have anti-inflammatory and hence anti-arthritic activity in humans.

TABLE 2
Anti-inflammatory activity of DPA using the mouse writhing test

| | |
|---|---|
| Number of | % inhibition of |
| writhes | writhing |

A. Subcutaneous injection, 6 hours before PBQ

| | | |
|---|---|---|
| Saline | 110 | — |
| DPA 500 mg/kg | 28 | 75 |
| Saline | 166 | — |
| DPA, 250 mg/kg | 106 | 36 |
| 62.5 mg/kg | 108 | 35 |

B. Oral administration of DPA, 2.5 hours before PBQ

| | | |
|---|---|---|
| Saline | 148 | — |
| DPA, 250 mg/kg | 133 | 10 |
| 500 mg/kg | 110 | 24 |
| 1000 mg/kg | 56 | 62 |

C. Intraperitoneal injection of DPA, 2 hours before PBQ

| | | |
|---|---|---|
| Saline | 125 | — |
| DPA, 250 mg/kg | 34 | 73 |
| 500 mg/kg | 13 | 90 |

3. Analgesic activity of DPA using the mouse hot plate test.

The mouse hot plate is a generally recognized method for evaluating analgesic drugs that act on the endorphin system, e.g., morphine-like drugs and drugs that increase endorphin levels, e.g., DPA. Such drugs are useful for treating many kinds of painful conditions including arthritis, headache, low back pain, musculoskeletal pain, pre-menstrual pain, and dental pain.

In this method, groups of mice (6 albino mice per group, weighing between 22 and 25 grams) are individually placed on a hot plate at 55° C. and a beaker is placed over the mouse. The time for the mouse to jump in an attempt to escape the painful condition is recorded. An analgesic substance is one which increases the threshold to pain thereby permitting the mouse to remain on the hot plate for longer times. The effect of the drug is compared to that of a saline control. DPA at different doses was injected intraperitoneally into the mice 2 hours before they were placed on the hot plate, and jumping time determined over the next 1-2 hours. The effect of DPA was compared with that of an injection of saline.

For studies involving combinations of drugs, the combination was injected at the same time. In the studies shown below, the second drug consisted of indomethacin, diclofenac or acetaminophen. Each of these drugs is used for treating arthritis as well as many other painful conditions.

TABLE 3
Analgesic activity of DPA, DPA plus indomethacin,
DPA plus diclofenac and DPA plus acetaminophen
using the mouse hot plate method

| | Increase in threshold to |
|---|---|
| | jumping compared to control |
| DPA, 250 mg/kg | 3-fold |
| Indomethacin, 20 mg/kg | 0 |
| DPA, 250 mg/kg PLUS | 11-fold |
| indomethacin, 20 mg/kg | |
| Diclofenac, 20 mg/kg | 0 |
| DPA, 250 mg/kg PLUS | 12-fold |
| diclofenac, 20 mg/kg | |

The importance of this experiment is the demonstration that the combination of DPA plus a NSAIDS such as indomethacin or diclofenac provides a greatly enhanced degree of analgesia. The analgesia produced is far greater than the sum of the individual drugs.

A similar result was obtained when DPA was combined with acetaminophen (Tylenol).

4. Efficacy of DPA in arthritis and a variety of other musculoskelatal diseases; human studies.

Forty-three patients were studied in an open-label, cross-over procedure. Of these, 32 had arthritis. All of the patients had previously been given many treatments without success in relieving their pain. Pre-DPA administration treatments used included such potent drugs as Percodan, Darvocet, Valium and aspirin as well as acupuncture. Prior to giving DPA 800 mg/day by mouth, all treatments were stopped. Patients were given a card in which they scored their degree of pain on a scale of 1-4: 1 indicated no relief, 2 approximately 25% relief, 3 approximately 50% relief, and 4 complete relief. Results are shown in Table 4.

TABLE 4
Efficacy of DPA in humans with arthritis and other skeletal diseases

| Complete | Excellent | Moderate | No relief of |
|---|---|---|---|
| relief of pain | relief of pain | relief of pain | pain |
| 2 | 7 | 19 | 15* |

*Most of these patients took DPA for only 1-2 weeks. They discontinued the DPA because of lack of success. For very severe conditions, DPA should be taken for longer periods of time and at even higher doses.

What these human results show is that DPA alone at the moderate dose of 800 mg/day could successfully decrease one of the prominent signs of these diseases, namely pain.

Pain is perhaps the most debilitating aspect of arthritis. In this invention, we propose to use DPA or DLPA along with other drugs or natural substances in order to enhance the analgesic efficacy of DPA in arthritis. Table 5 presents some suggested dosages for using DPA, or DLPA alone, or in combination with the various compounds discussed previously for treating arthritis and a variety of painful conditions in humans.

TABLE 5
Suggested dosage schedule for treating arthritis and other
painful conditions in humans using DPA, DLPA alone or
in combination with various other compounds

| Dose of DPA | Dose of DLPA | Dose of second component |
|---|---|---|
| 10-3000 mg/day | — | — |
| — | 20-6000 mg/day | — |
| 10-3000 mg/day | — | Glucosamine: |
| | | 1000-1500 mg/day |
| — | 20-6000 mg/day | Glucosamine: |
| | | 1000-1500 mg/day |
| 10-3000 mg/day | — | Chondroitin sulfate: |
| | 100-1000 mg/day | |
| — | 20-6000 mg/day | Chondroitin sulfate: |
| | 100-1000 mg/day | |
| 10-3000 mg/day | — | Aspirin: 85-3000 mg/day |
| | 20-6000 mg/day | Aspirin: 85-3000 mg/day |
| 10-3000 mg/day | — | Ibuprofen: |
| | | 200-2000 mg/day |
| — | 20-6000 mg/day | Ibuprofen: |
| | | 200-2000 mg/day |
| 10-3000 mg/day | — | Indomethacin: |
| | | 10-400 mg/day |
| | 20-6000 mg/day | Indomethacin: |
| | | 10-400 mg/day |

Similar combinations of DPA or DLPA with all other NSAIDs are herein proposed including the new COX 2 inhibitors. (See Table 6)

TABLE 6
List of NSAIDs, including COX 2 Inhibitors

| | Approximate doses |
|---|---|
| Name | mg/day |
| diclofenac | 25-300 |
| Etadolac | 100-1500 |
| fenoprofen | 300-4000 |
| fluriprofen | 100-600 |
| ibuprofen | 200-6000 |
| indomethacin | 10-400 |
| ketoprofen | 50-400 |
| ketorolac | 10-120 |
| meclofenamate | 100-800 |
| nabumetone | 250-4000 |
| naproxen | 100-3000 |
| oxaprozin | 600-3000 |
| piroxicam | 5-40 |
| sulindac | 50-1000 |
| tolmetin | 400-4000 |
| rofecoxib | 10-150 |
| celecoxib | 100-400 |

TABLE 7
List of Nutraceuticals to be combined with DPA/DLPA
all Weight Percentages About 5% —about 90%)

| Name | Approximate doses | |
|---|---|---|
| Glucosamine | 500-2000 | mg/day |
| Chondroitin Sulfate | 400-1000 | mg/day |
| Cats Claw (Uncaria tomentosa) | 20-100 | mg/day |
| Devils Claw (Harpagophytum | 400-600 | mg/day |
| procumbens) | | |
| Cetyl Myristoleate (CMO) | 100-800 | mg/day |
| Cetyl Esters with CMO | 100-800 | mg/day |
| Coenzyme Q-10 | 60-200 | mg/day |
| Fructose 1.6 diphosphate | 2-10 | g/day |
| Glutathione | 100-700 | mg/day |
| Melatonin | 3-10 | mg/day |
| Kava Kava Extract, 30% | 200-800 | mg/day |
| SAM-e | 400-1200 | mg/day |
| Bromelain | 60-160 | mg/day |
| White Willow Bark powder and | 100-500 | mg/day |
| extract 0.05-.99% Salicin | | |
| Type II Collagen | 100-2400 | mg/day |
| Methyly-sulfonyl-methane | 500-2500 | mg/day |
| (MSM) | | |

### SCOPE OF THE INVENTION

The above presents a description of the best mode contemplated of carrying out the present invention, and of the manner and process of making and using it, in such full, clear, concise, and exact terms as to enable any person skilled in the art to which it pertains to make and use this invention. This invention is, however, susceptible to modifications and alternate constructions from that discussed above which are fully equivalent. Consequently, it is not the intention to limit this invention to the particular embodiments disclosed. On the contrary, the intention is to cover all modifications and alternate constructions coming within the spirit and scope of the invention as generally expressed by the following claims, which particularly point out and distinctly claim the subject matter of the invention:

1-11. Cancel

12. A composition comprising a therapeutic amount of phenylalanine and a therapeutic amount of SAMe.

13. The composition according to claim 12 where the SAMe includes as a major component the (SS)-(+)-SAMe.

14-57. Cancel

58. A composition comprising a therapeutic amount of leucine and a therapeutic amount of SAMe.

59. The composition according to claim 58 where the SAMe includes as a major component the (SS)-(+)-SAMe.

60-103. Cancel

104. A composition comprising a therapeutic amount of hydrocinnamic acid and a therapeutic amount of SAMe.

105. The composition according to claim 104 where the SAMe includes as a major component the (SS)-(+)-SAMe.

106-144. Cancel

145. The composition according to claim 12 including L-tryptophine.

146. The composition according to claim 12 including glutathione.

147. The composition according to claim 58 including L-tryptophine.

148. The composition according to claim 58 including glutathione.

149. The composition according to claim 104 including L-tryptophine.

150. The composition according to claim 104 including glutathione.
